SEQUENCE LISTING (4 sequences)

SEQ ID NO: 1 - RNA, 23 nt, synthetic construct
  source 1..23: mol_type = other RNA
caaagactgcaattacttttgcg

SEQ ID NO: 2 - RNA, 22 nt, synthetic construct
  source 1..22: mol_type = other RNA
agctacagttacttttgcacca

SEQ ID NO: 3 - RNA, 81 nt, synthetic construct
  source 1..81: mol_type = other RNA
attaggatggtgcaaaagtaatgtggtttttttctttacttttaatggcaaagactgcaattacttttgcgccaacctaat

SEQ ID NO: 4 - RNA, 80 nt, synthetic construct
  source 1..80: mol_type = other RNA
aatactaggtttgagcaaaagtaattgcggttttgccatcatgccaaaagctacagttacttttgcaccagcctaatatt

(19) United States
(12) Patent Application Publication
(10) Pub. No.: US 2024/0401041 A1
(43) Pub. Date: Dec. 5, 2024

(54) METHODS FOR IMPROVING RELAXATION OF STRIATED MYOCYTES

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); SORBONNE UNIVERSITÉ, Paris (FR); UNIVERSITÉ PARIS CITÉ, Paris (FR)

(72) Inventors: Jean-Sébastien HULOT, Paris Cedex 15 (FR); Eva VERMERSCH, Versailles (FR)

(21) Appl. No.: 18/575,442

(22) PCT Filed: Jul. 7, 2022
(86) PCT No.: PCT/EP2022/068936

§ 371 Date: Dec. 29, 2023

(30) Foreign Application Priority Data
Jul. 9, 2021 (EP) 21305954.6

(51) Int. Cl. C12N 15/113

(52) U.S. Cl. CPC C12N 15/113; C12N 2310/141; C12N 2320/31

(57) ABSTRACT

The Inventors developed conditions allowing to efficiently detect differences in cardiomyocytes relaxation phases associated with increased cardiomyocytes stiffness. They used a library of patient-specific human induced pluripotent stem cells (hiPSC) either from healthy donors or carrying mutations (i.e., MYH7 and BRAF mutations) associated with hypertrophic cardiomyopathy, a condition typically associated with impaired diastolic function as well as an increase in cardiomyocytes passive stiffness. They performed a high throughput screening on hiPSC-derived cardiac cells to identify microRNAs capable of modifying the relaxation rates of cardiomyocytes. In particular, they set up a large-scale functional genomics using miRNAs screening. All identified miRNAs were tested for their impact on cardiac cells movement and calcium transient. miRNAs with the highest impact were in particular tested on ECTs and changes in diastolic function, were measured and compared to the results obtained at the cell level. They manipulated the most interesting ‘hits’ in 3D models using similar readouts as in primary assays. They tested the impact of the positive ‘hits’ in mechanical models (developed during the exploratory part) and establish physiological and biochemical mechanisms of action of the identified key proteins. They finally identified two promising miRNAs that could be used for improving striated myocytes relaxation and, more generally, to alleviate symptoms related to striated muscle stiffness, in particular in the context of heart failure with a preserved ejection fraction (HFpEF).

### SEQUENCE LISTING

This application includes as the Sequence Listing the complete contents of the XML file “SequencesXML_21052 PCT”, created Aug. 16, 2024, containing 8,674 bytes, hereby incorporated by reference.

### FIELD OF THE INVENTION

The present invention is in the field of medicine, in particular myology.

### BACKGROUND OF THE INVENTION

Left ventricular (LV) diastolic function plays an important role in cardiac performance and is mainly determined by the efficiency of myocardial relaxation. In the healthy human myocardium, the velocity of myocardial relaxation directly influences the ability to fill the LV while keeping low filling pressures (1, 2). In response to a higher demand, such as during exercise, relaxation speed is increased in order to accelerate diastolic LV filling despite a shortening of the time available for ventricular filling with tachycardia (3, 4). Reciprocally, an impaired diastolic reserve, measured as an inadequate increase in myocardial relaxation velocity, is considered a hallmark of heart failure (notably for heart failure with preserved ejection fraction (HFpEF)) and is associated with a progressive decline in exercise capacity (2, 4-6). Theoretically, pharmacological agents that facilitate myocardial relaxation would improve LV compliance and would be ideal for the treatment of diastolic dysfunction. However, our understanding of the mechanisms regulating myocardial relaxation is limited, especially in human.

Myocardial relaxation is a complex multi-component process which, at least in part, depends on the ability of cardiomyocytes to relax (i.e., lusitropy). After each contraction, cardiomyocytes exhibit a non-linear viscoelastic behavior as they rapidly return to their original configuration without memory of the mechanical compaction induced by the contraction. In addition, the stretching of the cardiomyocytes (within the left ventricular walls) as the heart fills with blood during diastole invokes considerable viscoelastic forces (7, 8). In addition to calcium cycling influence, it has been proposed that the rapid elastic response of cardiomyocytes depends on elements composing the myofilament and the cytoskeleton. For instance, the giant protein titin is an important determinant of myofilament diastolic tension (9, 10) and a contributor of viscous forces (11). Changes in titin phosphorylation modifies its compliance, which is commonly altered in diseases with lower diastolic compliance (12). Recent data have also shown the importance of the non-sarcomeric cytoskeleton (consisting of microtubules and desmin intermediate filaments) in cardiomyocytes viscoelasticity. The post-translational detyrosination of microtubules influences the stability of the microtubules network and promotes its cross-linking with the myocyte cytoskeleton and intermediate filament network (13, 14). Desmin intermediate filaments act as elastic elements surrounding the myofilament Z-disc. In heart failure, there is an increased abundance of detyrosinated microtubules and desmin intermediate filaments (15, 16). Mutations in DESMIN can lead to restrictive cardiomyopathy which is primarily characterized by impaired relaxation and diastolic dysfunction (17, 18).

It is likely that the multi-scale remodeling of these elements in heart disease (and particularly in HFpEF) leads to abnormal myocardial viscoelasticity and a disturbed ventricular compliance which directly impede diastolic filling. Yet, our understanding of myocardial relaxation and its regulation remain incomplete. Techniques and methods to characterize myocardial viscoelasticity at the tissue and the cell-levels have only recently emerged and the field is globally understudied, especially in human cardiomyocytes. Similarly, the impact of the cardiomyocyte viscoelastic properties in the physiology of cardiac performance is not understood, especially in human. MicroRNAs (miRs) are endogenous 22-nucleotide single stranded RNAs that can bind and suppress multiple messenger RNAs. It is estimated that miRNAs control almost every cellular process and 60% of the proteome (19). Hence, miRNAs library is an attractive tool to identify regulators of a specific phenotype within a phenotypic screening strategy (20).

Here we set out to systematically identify microRNAs (miRs) enhancing cardiomyocyte (CM) relaxation using a synthetic miRNA library of human origin applied to human models based on human induced pluripotent stem cells derived cardiomyocytes (hiPSC-CMs).

### SUMMARY OF THE INVENTION

The present invention is defined by the claims. In particular, the present invention relates to the use of miR-548u, miR-548v or a precursor thereof for improving striated myocytes relaxation.

### DETAILED DESCRIPTION OF THE INVENTION

The Inventors developed conditions allowing to efficiently detect differences in cardiomyocytes relaxation phases associated with increased cardiomyocytes stiffness. They used a library of patient-specific human-induced pluripotent stem cells (hiPSC). They performed a high throughput screening on hiPSC-derived cardiac cells to identify microRNAs capable of modifying the relaxation rates of cardiomyocytes. All identified miRNAs were tested for their impact on cardiac cells movement and calcium transient. They manipulated the most interesting ‘hits’ in engineered cardiac tissues (3D models) using similar readouts as in primary assays. They tested the impact of the positive ‘hits’ in mechanical models (developed during the exploratory part) and establish physiological and biochemical mechanisms of action of the identified key proteins. They finally identified two promising miRNAs that could be used for improving striated myocytes relaxation and, more generally, to treat striated muscle stiffness, in particular in the context of heart failure with a preserved ejection fraction (HFpEF). These two miRNAs are miR-548u and miR-548v.

The first object of the present invention relates to a method for improving striated muscle cell relaxation in a subject in need thereof comprising administering a therapeutically effective amount of at least one miRNA selected from the group consisting of miR-548u and miR-548v.

As used herein, the term “subject” or “patient” denotes a mammal, in particular humans. Typically, a subject according to the invention refers to any subject afflicted with or susceptible to be afflicted with striated myocytes stiffness. In a particular embodiment, the subject is afflicted with or susceptible to be afflicted with cardiomyocytes stiffness, in particular in the context of heart failure with preserved ejection fraction.

As used herein, the term “myocyte” or “muscle cell” has its general meaning in the art and denotes a contractile and excitable cell. In particular, myocyte comprise essentially myofibrils made up of myofilaments of actin and myosin. Actin filaments are organized into a dynamic network that change shape according to internal or external constraints. Myosin is a motor protein involved in the muscle contraction via actin network. More precisely, muscle contraction corresponds to a shortening of sarcomeres (i.e. contractile functional unit of striated muscular fibril) due to a relative sliding of actin and myosin filaments.

As used herein, the term “striated myocyte” or “striated muscle cell” has its general meaning in the art and denotes cardiac cells, also named cardiomyocytes, or skeletal cells, also named rhabdomyocytes. These cells contain many sarcosomes (i.e. a specialized mitochondrion occurring in a muscle fibril) in order to generate sufficient ATP since these cells have high energy requirements. Striated muscle cells form striated muscles, highly organized tissues converting energy to physical work to generate force and to contract to support movements such as respiration, locomotion and posture, or to pump blood throughout the body. Striated muscles are so called because of their sarcomeres which are structurally arranged in regular bundles. Striated muscles are myocardium or skeletal muscle.

As used herein, the term “striated muscle relaxation” denotes a state when striated myocytes have a low resting tension. An abnormal relaxation state can lead to an abnormal muscle stiffness, due as example, to an abnormal ionic gradient, a dysfunctional channel or an abnormal transporter concentration, or to an abnormal myocytes rigidity due, as example, to an abnormal microtubule polymerization or dynamic, an abnormal post-translational microtubule modification, an abnormal titin phosphorylation, a shorter or stiffer isoforms of titin and more generally to every causes leading to a loss of viscoelastic properties of striated myocytes or to a high resting tension of striated myocytes. As example, such relaxation may be assessed with impulse elastography, myostretching or atomic force microscopy.

As used herein, the expression “improve striated muscle relaxation” refers to an improvement in the striated muscle relaxation that can be at least about 10%, e.g., at least about 20%, 30%, 40%, 50%, 60%, 70%, 80%, 90%, 95%, 96%, 97%, 98%, 99%, or more.

The present method of the present invention is thus particularly suitable for the treatment of muscle stiffness caused by prolonged immobility secondary to disease, orthopedic injury, neurologic causes of paralysis such as stroke, traumatic brain injury, multiple sclerosis, spinal cord injury, cerebral palsy or developmental causes of contractures, such as specific subtypes of arthrogryposis multiplex congenita, as well as muscle pain and joint stiffness from non-neurologic causes such as from prolonged bed rest, post-operative stiffness, myofascial pain and fibromyalgia, over-use, repetitive trauma, age-related muscle stiffness and muscle-stiffness due to diabetes. More particularly, the method of the present invention is suitable for the treatment of spasticity that is a common secondary disabling condition following many neurological disorders such as stroke, cerebral palsy, spinal cord injury, and multiple sclerosis. Even more particularly, the method of the present invention is suitable for the treatment of striated muscle stiffness that is induced by Parkinson's disease, tetanus, muscle tetany, myotonia, dystonia, spasmophily, sclerosis, myofascial pain syndrome, myalgia, polymyalgia rheumatica, fibromyalgia, meningitis, lupus, mononucleosis or Lyme's disease.

In particular, the method of the present invention is particularly suitable for improving cardiomyocyte relaxation. As used herein, the term “cardiomyocyte” has its general meaning in the art and denotes the muscular cells (i.e. myocytes) that make up the cardiac muscle, the myocardium. Cardiomyocytes are linked together by intercalated discs and every cardiomyocyte is able to proceed with spontaneous rhythmic depolarization. This ability to be polarized/depolarized implies a cardiac action potential, consisting in two alternatives cycles: systole when cells are depolarized (contraction) and diastole when cells are repolarized (relaxation).

The method of the present invention is thus particularly suitable for the treatment of heart failure with preserved ejection fraction (HFpEF).

As used herein, the term “heart failure with preserved ejection fraction” or “HFpEF” has its general meaning in the art and refers to a complex syndrome characterized by heart failure (HF) signs and symptoms and a normal or near-normal left ventricular ejection fraction (LVEF). More specific diagnostic criteria include signs/symptoms of HF, objective evidence of diastolic dysfunction, disturbed left ventricular (LV) filling, structural heart disease, and elevated brain natriuretic peptides. Additional cardiac abnormalities can include subtle alterations of systolic function, impaired atrial function, chronotropic incompetence, or haemodynamic alterations, such as elevated pre-load volumes. The term is also referred to as diastolic heart failure. Three main steps could be used to diagnose HFpEF (Yancy et al., 2013):
Clinical signs or symptoms of heart failure;
Evidence of preserved or normal left ventricle ejection fraction (>45-50%); and
Evidence of abnormal left ventricle diastolic dysfunction.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “miRNA” denotes a small single-strain non-coding RNA molecule. miRNAs are involved in post-transcriptional regulation of gene expression in multicellular organisms. miRNAs are at least partially complementary to one or more mRNA to downregulate gene expression by inducing translational repression, mRNA cleavage or deadenylation.

As used herein, the term “miR-548u” denotes a miRNA able to improve striated myocyte relaxation as demonstrated in the present invention. MiR-548u is encoded by MIR548U gene (HGNC: 38316; Entrez Gene: 100422884; ENSEMBL: ENSG00000212017; miRBase: MI0014168) located in chromosome 6. In particular, the term “miR-548u” refers to the mature miR-548u sequence and homologs, variants, and isoforms thereof. The mature sequence of miR-548u is represented by SEQ ID NO:1.

> hsa-miR-548u MIMAT0014168
SEQ ID NO: 1
CAAAGACUGCAAUUACUUUUGCG

As used herein, the term “miR-548v” denotes a miRNA able to improve striated myocyte relaxation as demonstrated in the present invention. MiR-548v is encoded by MIR548V gene (HGNC: 38302; Entrez Gene: 100422850; ENSEMBL: ENSG00000265520; miRBase: MI0014174) located in chromosome 8. In particular, the term “miR-548v” refers to the mature miR-548v sequence and homologs, variants, and isoforms thereof. The mature sequence of miR-548v is represented by SEQ ID NO:2.

> hsa-miR-548v MIMAT0015020
SEQ ID NO: 2
AGCUACAGUUACUUUUGCACCA

The methods described herein can include the use of nucleotide sequences of miR-548u, miR-548v or a precursor thereof, or a variant that comprise a nucleotide sequence at least about 80%, 85%, 90%, 95%, 98%, 99% or more identical to the nucleotide sequence of miR-548u, miR-548v or a precursor thereof. Those of skill in the art readily understand how to determine the identity of two nucleic acid sequences. For example, the identity can be calculated after aligning the two sequences so that the identity is at its highest level. Sequence identities can also be obtained for nucleic acids by, for example, the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989, which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity and to be disclosed herein.

Administration of miRNAs can occur via multiple routes. miRNAs can be chemically synthesized and administered to the cell, or miRNAs can be encoded in a nucleic acid sequence that is expressed in the cell via a DNA-based expression vector.

A chemically synthesized miRNA can comprise a single-stranded RNA (ssRNA) or a double-stranded RNA (dsRNA) molecule. The RNA molecule can comprise the pri-miRNA, which can be hundreds of nucleotides in length, a pre-miRNA, which is generally 60-80 nucleotides in length, or the mature miRNA, which is generally 18-23 nucleotides in length. Administration of the pri-miRNA and pre-miRNA to the cell results in production of the mature miRNA. RNA molecules can be synthesized in vitro from a DNA template, or can be synthesized commercially and are available from such corporations as Dharmacon, Inc. (Lafayette, Colo.), Qiagen (Valencia, Calif.), and Ambion (Austin, Tex.). In some embodiments, the miRNA is a synthetic miR-548u or miR-548v duplex that mimics respectively pre-miR-548u or pre-miR-548v. In some embodiments, the miRNA is miR-548u and comprises the stem loop sequence as set forth in SEQ ID NO:3.

> hsa-mir-548u MI0014168
SEQ ID NO: 3
AUUAGGAUGGUGCAAAAGUAAUGUGGUUUUUUUCUUUACUUUUAAUGGC
AAAGACUGCAAUUACUUUUGCGCCAACCUAAU

In some embodiments, the miRNA is miR-548v and comprises the stem loop sequence as set forth in SEQ ID NO:4.

>hsa-mir-548v MI0014174
SEQ ID NO: 4
AAUACUAGGUUUGAGCAAAAGUAAUUGCGGUUUUGCCAUCAUGCCAAAAG
CUACAGUUACUUUUGCACCAGCCUAAUAUU

The methods described herein can use both miRNA and modified miRNA derivatives, e.g., miRNAs modified to alter a property such as the specificity and/or pharmacokinetics of the composition, for example, to increase half-life in the body, e.g., crosslinked miRNAs. Thus, the invention includes methods of administering miRNA derivatives that include miRNA having two complementary strands of nucleic acid, such that the two strands are crosslinked. The oligonucleotide modifications include, but not limited to, 2′-O-methyl, 2′-fluoro, 2′-O-methyoxyethyl and phosphorothiate, boranophosphate, 4′-thioribose. (Wilson and Keefe, Curr. Opin. Chem. Biol. 10:607-614 (2006); Prakash et al., J. Med. Chem. 48:4247-4253 (2005); Soutschek et al., Nature 432:173-178 (2004)).

In some embodiments, the miRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying miRNA derivatives in this way may improve cellular uptake or enhance cellular targeting activities of the resulting miRNA derivative as compared to the corresponding miRNA, are useful for tracing the miRNA derivative in the cell, or improve the stability of the miRNA derivative compared to the corresponding miRNA. The miRNA nucleic acid compositions can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al., Drug Deliv. Rev. 47(1):99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

Alternatively nucleic acid molecules encoding the miRNA of the present invention may be used. Nucleic acid molecules encoding miRNAs are useful, e.g., where an increase in the expression and/or activity of a miRNA is desirable. Nucleic acid molecules encoding miR-548u or miR-548v, optionally comprising expression vectors, can be used, e.g., for in vivo or in vitro expression of a selected miRNA. In some embodiments, expression can be restricted to a particular cell types so as to reconstitute the function of the selected miRNA in a cell, e.g., a cell in which that miRNA is misexpressed. A nucleic acid encoding the selected miRNA can be inserted in an expression vector, to make an expression construct. A number of suitable vectors are known in the art, e.g., viral vectors including recombinant retroviruses, adenovirus, adeno-associated virus, and herpes simplex virus-1, adenovirus-derived vectors, or recombinant bacterial or eukaryotic plasmids. For example, the expression construct can include: a coding region; a promoter sequence, e.g., a promoter sequence that restricts expression to a selected cell type (i.e., a myocyte-specific promoter or a cardiomyocyte-specific promoter, such as MEF2 promoter or cTnT promoter respectively), a conditional promoter, or a strong general promoter; an enhancer sequence; untranslated regulatory sequences, e.g., a 5′-untranslated region (5′-UTR), a 3′-UTR; a polyadenylation site; and/or an insulator sequence. Such sequences are known in the art, and the skilled artisan would be able to select suitable sequences. See, e.g., Current Protocols in Molecular Biology, Ausubel, F. M. et al. (eds.) Greene Publishing Associates, (1989), Sections 9.10-9.14 and other standard laboratory manuals.

In clinical settings, the nucleic acids encoding miR-548u or miR-548v can be introduced into a patient by any of a number of methods known in the art. For instance, a pharmaceutical preparation comprising the nucleic acid delivery system can be introduced systemically, e.g. by intravenous injection, and specific transduction of the miRNA in the target cells occurs predominantly from specificity of transfection provided by the gene delivery vehicle, cell-type or tissue-type expression due to the transcriptional regulatory sequences controlling expression of the miRNA, or a combination thereof. In some embodiments, initial delivery of the miRNA is more limited with introduction into the animal being quite localized. For example, the miRNA delivery vehicle can be introduced by catheter (see U.S. Pat. No. 5,328,470) or by stereotactic injection (e.g. Chen et al. (1994) PNAS 91: 3054-3057).

A used herein, the term “therapeutically effective amount” above described is meant a sufficient amount of the compound of miR-548u or miR-548v for achieving a therapeutic effect (reducing striated myocyte stiffness by improving striated myocyte relaxation). It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved.

In some embodiments, the miRNA of the present invention is administered in combination with at least one other therapeutic agent such as a muscle relaxant such as atracurium besilate, baclofene, carisoprodol, cisatracurium besilate, dantrolene, mivacurium chlorure, methocarbamol, pancuronium bromure, rocuronium bromure, suxamethonium, thiocolchicoside, tizanidine, tetrazepam or vecuronium bromure. Others examples of at least one other therapeutic agent may be Angiotensin Converting Enzyme Inhibitors (ACEIs), angiotensin, Aldosterone Receptor Antagonists (ARDs) or β-blockers. These therapeutic agents are usually used in the context of heart failure with preserved ejection fraction. Others examples of at least one other therapeutic agent may be dopamine precursors, dopamine agonists such as apomorphine or rotigotine or inhibitor of dopamine precursor degradation such as Catechol-O-Methyltransferase inhibitors or Monoamine oxidase inhibitors. These therapeutic agents are usually used in the context of Parkinson's disease.

A further aspect of the invention relates to a therapeutic composition comprising at least one miRNA selected from the group consisting of miR-548u or miR-548v for improving striated muscle relaxation in a subject in need thereof.

Typically, the miR-548u or miR-548v may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Galenic adaptations may be done for specific delivery in the small intestine or colon. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising miR-548u or miR-548v of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. MiR-548u or miR-548v of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifusoluble agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. Multiple doses can also be administered. In addition to the miR-548u or miR-548v of the invention formulated for parenteral administration, such as intravenous or intramuscular injection, other pharmaceutically acceptable forms include, e.g. tablets or other solids for oral administration; liposomal formulations; time release capsules; and any other form currently used.

The invention will be further illustrated by the following figures and examples. However, these examples and figures should not be interpreted in any way as limiting the scope of the present invention.

### FIGURES

FIG. 1: Level of expression of hsa-miRNA-548u (1) and hsa-miR-548v (2) in ECT transfected with miR negative (first two columns), miR 548u (third and fourth column) and miR-548v (fifth and sixth column).

FIG. 2: Spontaneous vs paced beating frequency (Hz) of ECT transfected with miR negative (1), miR 548u (2) or miR 548v (3).

FIG. 3: Force (N) developed by ECT without electrical stimulation (A) or with an applied pacing frequency at 0.6 Hz (B). A relative force of ECT (before transfection vs after transfection) was calculated to normalize (C).

FIG. 4: Mean relaxation velocity of ECT without stimulation (A) or with an applied frequency at 0.6 Hz (B). A relative mean relaxation velocity of ECT (before transfection vs after transfection) was calculated to normalize (C).

FIG. 5: (A) Mean relaxation velocity, mean contraction velocity and peak amplitude of motion in hiPSC-derived cardiomyocytes transfected with hsa-miR-548v or miR negative control. (B) Representative records of beat-to-beat motion (left) and averaged contraction/relaxation cycle (right) recorded from cardiomyocytes transfected with hsa-miR-548v or miR negative control.

FIG. 6: (A) Evaluation of hsa-miR-548v expression levels 3 days after transfection. (B) Representative records of beat-to-beat motion from hECTs transfected with hsa-miR-548v or miR negative control. (C) Averaged contraction/relaxation cycle recorded from ECTs transfected with hsa-miR-548v or miR negative control. (D) Relative mean relaxation velocities in hECT 3 days after transfection with hsa-miR-548v or miR negative control. (*p<0.05). (E) Relative developed force in hECT 3 days post transfection with hsa-miR-548v or miR negative control.

FIG. 7: (A) Amplitude of the calcium transient of hiPSC-CM 3 days after transfection with hsa-miR-548v or miR negative control. (B) Rising slope of the calcium transient of hiPSC-CM 3 days after transfection with hsa-miR-548v or miR negative control. (C) Falling slope of hiPSC-CM 3 days after transfection with hsa-miR-548v or miR negative control.

FIG. 8: Representative quantification of detyrosinated alpha-tubulin and GAPDH in hiPS-CM transfected with hsa-miR-548v or miR negative control.

FIG. 9: (A) Staircase protocol, each increment represents a strain of 6 m (5% stretch). (B) Force measurements at different stretch levels and derived parameters. (C) Mechanical response of hiPSC-CMs transfected with hsa-miR-548v and miR negative control to different stretch levels. Left: Peak stress (viscous and elastic stress); Middle: steady state stress (elastic stress); Right: Relaxation stress (viscous response)

### EXAMPLE 1

### Material and Methods

### Differentiation

Induced pluripotent stem cells (iPSC) were differentiated by a 2D differentiation protocol. The protocol used is adapted from Sharma et al. (Sharma et al., 2015). Briefly, when B6 dishes reached 80% confluency, iPSC colonies were dissociated with ReLeSR™ (Stemcell, 05873) and seeded on Matrigel® (Corning, 354277) coated 12-well culture plates in mTeSR™1 culture medium (Stemcell, 85850). IPS were next cultured until 80% to 90% confluency and then change to RPMI 1640 (ThermoFisher, 72400054)+B27 supplement minus insulin (ThermoFisher, A1895601) medium and 6 M CHIR99021 (Abcam, ab120890) medium for 48 hr. On day 2, the CHIR-containing culture medium is changed with RPMI/B27 without insulin medium for 24 h. At day 3, the media is changed to RPMI/B27 without insulin with 5 M Wnt inhibitor IWR1 (Sigma, I0161-5MG) until day 5. At day 5, the medium is changed back to RPMI/B27 without insulin for 48 hours. At day 7, cells were cultured in RPMI+B27 with insulin (ThermoFisher, 17504044) and medium was changed day 9 with the same medium. On day 11 post-differentiation, the medium in each well is changed to low glucose medium (B27 Supplement into glucose-free RPMI 1640 (ThermoFisher, 11879020)) for 3 days. On day 14, cells were dissociated into single cells using enzyme T (Miltenyi, 130-110-204) and seeded into a new Matrigel® coated 12-well plate (approximately 1.2E6 cells/well). On day 15, the medium was changed back to low glucose medium for a second glucose deprivation cycle for 3 more days. Most of the non-cardiomyocytes will die in this low-glucose culture condition. From day 18 onwards, cells were cultured in RPMI/B27 medium with insulin. The remaining cells will be highly purified cardiomyocytes.

### Engineered Cardiac Tissue (ECT)

To make an engineered cardiac tissue, we prepare a mix of 100 μL of cells/collagen/matrigel of 1:8:1 (v/v/v). On day 22, IPS-CM were dissociated using enzyme T. Simultaneously, we dissociated normal human dermal fibroblasts, and mixed IPS-CM with Fibroblasts at a ratio of 4:1 in RPMI+20% FBS (ThermoFisher, 10500064). We centrifuged and resuspended cells to obtain a concentration of 1.2E8 cardiomyocytes/mL. We prepared a 2.6 mg/mL iced-cold rat collagen I mix: 100 μL HEPES (Sigma H0887), 100 μL MEM 10× (Sigma, M9288) and 800 L 3.25 mg/mL collagen I (Sigma, 08-115). Finally, to prepare 10 ECTs, we mixed 100 μL of cells, 800 μL of rat collagen I mix (final concentration 2.5 mg/ml) and 100 μL of 9 mg/ml Matrigel (Corning, 356231) and filled 100 μL into each PDMS mold (K. Costa's Lab (Turnbull et al., 2014)). Then we incubated at 37° C., 5% CO2 for 2 h to allow the collagen to polymerize. We maintained in culture with halfmedium exchanges every two-days with high-glucose Dulbecco's modified Eagle medium (DMEM) supplemented with 10% FBS and 1% penicillin/streptomycin. Inserts in the casting mold were removed at 48 h.

### Transfection

After 13 days of tissue culture, ECT were recorded and transfected with micro-RNA. Transfection were performed with lipofectamine RNAimax (Invitrogen, 13778-150) with 25 nM of each miR referenced in Table 1. Media was changed 24 h hours post transfection and ECT movements were recorded 3 days after transfection (day 16 of ECT). ECT were compared to themselves in order to calculate a normalized relative response.

### Relaxation Phase Characterization

We used the flexible polydymethyl siloxane (PDMS) posts as force sensor, we recorded the movement and bending of the posts with a LabView script developed by K.Costa's lab. From the movement signal we extracted, amplitude of displacement, frequency and velocity using a custom made Matlab application. The developed force is estimated using the beam-bending equation from elasticity theory:

$$F = \frac{3 π {ER}^{4}}{2 {a}^{2} ( 3 L - a )} δ$$

### Where:

F is the tissue contraction force;
E, R, L respectively stand for the Young's modulus (1.33 MPa), radius (0.5 mm), and length of the PDMS posts (3.5 mm);
a is the height of the tissue on the post; 6 is the measured tip deflection.

These signals were obtained with ECT maintained in the original mold on a 37° C. warming plate with or without electrical filed stimulation.

qPCR

ECT were directly dry frozen after records in order to extract RNA. RNA were extracted using miRNAeasy mini Kit (Qiagen, 217004). cDNA synthesis and qPCR were performed using the miRCury LNA miRNA SYBR Green PCR RT kit (Qiagen, 339340). Primers used are listed in Table 2.

### Results

### Level of Expression of Hsa-miRNA-548u and Hsa-miR-548v in Transfected ECT

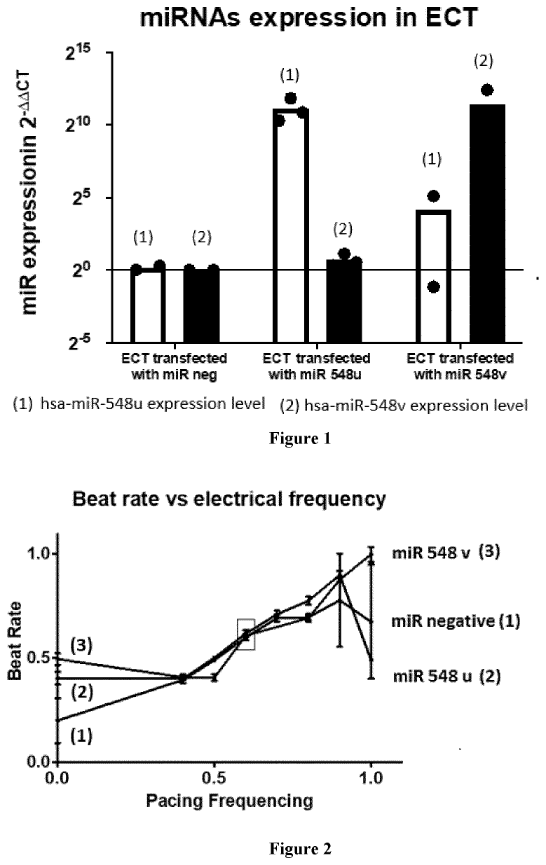

In ECT transfected with miR negative control, a minimal expression of miR-548u and miR-548v was assessed. When transfected with miR-548u, ECT demonstrate a significant and specific over-expression of miR-548u. When transfected with miR-548v, ECT demonstrate a significant over-expression of miR-548v and a slightly increased miR-548u expression. (FIG. 1).

### Spontaneous Vs Paced Beating Frequency of Transfected ECT

As demonstrated in FIG. 2, the only frequency where all three ECTs bate as the prescribed pacing is 0.6 Hz.

### Force Developed by ECT

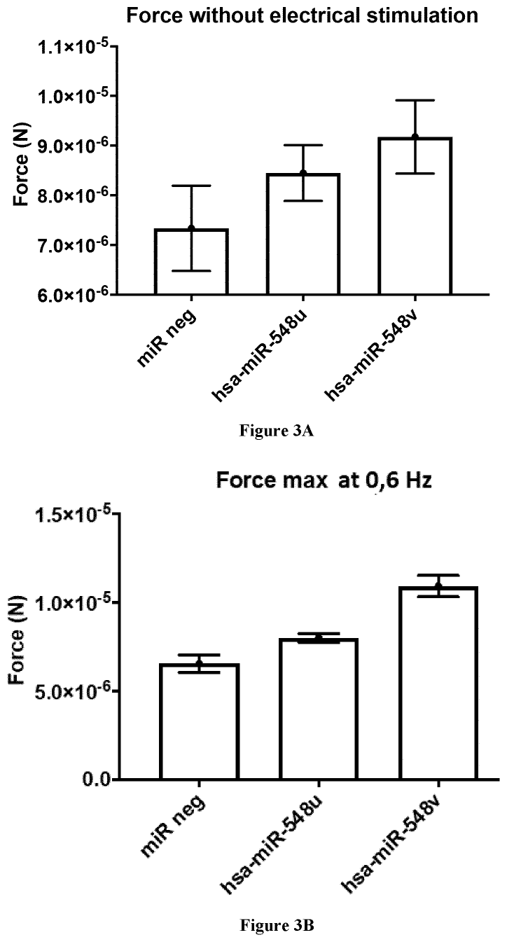

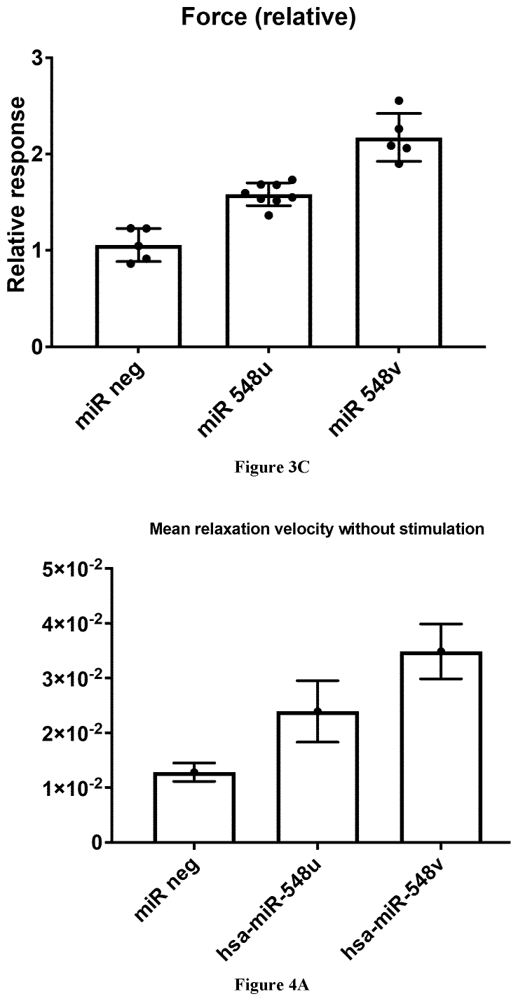

ECT were transfected with a negative miR, miR-548u or miR-548v. The ECT transfected with miR-548u or miR-548v demonstrated an increase in the developed force as compared to ECT transfected with miR negative (FIG. 3A,B,C).

### Mean Relaxation Velocity of ECT

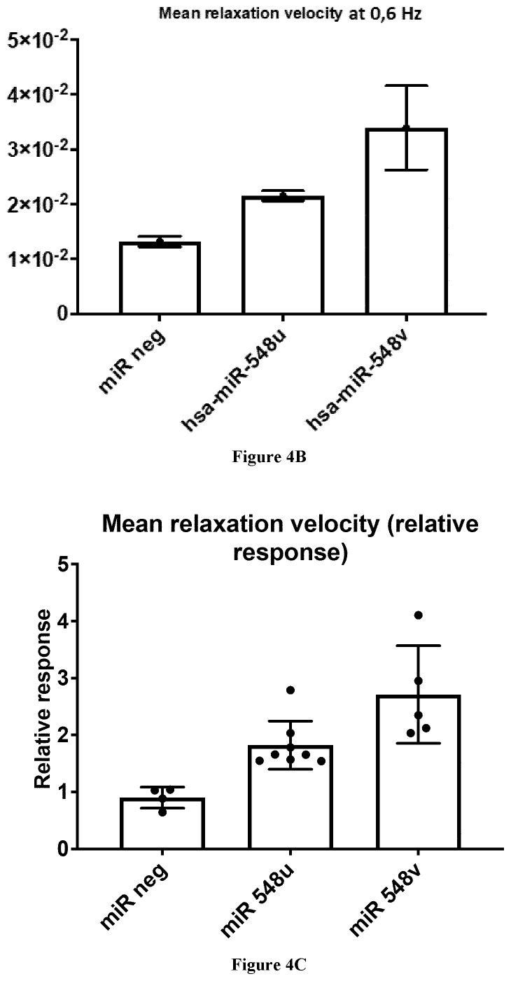

ECT were transfected with a negative miR, miR-548u or miR-548v. The ECT transfected with miR-548u or miR-548v demonstrated an increase in the relaxation velocity as compared to ECT transfected with miR negative, with the maximal amplitude observed for miR-548v (FIG. 4A,B,C).

### Conclusion

Both miR-548u and miR-548v demonstrate similar results, whereas their biochemical pathway seems different. According to in silico analysis, miR-548u appears to affect the control of microtubule dynamic, whereas miR-548v seems influence calcium transient, especially by impacting cationic transporters (data not shown). In one hand, miR-548u demonstrates an increased relaxation velocity and an improvement of contractive force. In another hand, miR-548v also demonstrates an increased relaxation velocity with an increased tissue contraction force. By improving striated muscle cell relaxation, miR-548u and miR-548v could be used for the treatment of striated muscle stiffness, more particularly in the context of heart failure with a preserved ejection fraction (HFpEF).

### Tables:

TABLE 1
miRNA used to perform transfection with lipofectamine
RNAimax (Invitrogen, 13778-150)

| miRVana miRNA mimic | Manufacturer | Catalog number | ID |
|---|---|---|---|
| Negative control #1 | ThermoFisher | 4464058 | — |
| hsa-miR-548u | ThermoFisher | 4464066 | MC18308 |
| hsa-miR-548v | ThermoFisher | 4464066 | MC18720 |

TABLE 2
primers used to perform the cDNA synthesis and qPCR using the
miRCury LNA miRNA SYBR Green PCR RT kit (Qiagen, 339340)

| Product Name | Manufacturer | Catalog number |
|---|---|---|
| RNU1A1 | Qiagen | YP00203909 |
| hsa-miR-548u | Qiagen | YP02104219 |
| hsa-miR-548v | Qiagen | YP02107607 |

### EXAMPLE 2

The inventors set out to systematically identify microRNA (miRs) enhancing cardiomyocyte (CM) relaxation using a synthetic miRNA library of human origin applied to human models based on human induced pluripotent stem cells derived cardiomyocytes (hiPSC-CMs).

### Material and Methods

High-Throughput Imaging Assays Using hiPSC-CMs

We used commercially available human pluripotent stem cell-derived cardiomyocytes (hiPSC-CM) from FUJIFILM Cellular Dynamics (iCell® cardiomyocytes²). Cells were thawed according to the manufacturer's recommendations in 384-wells plates (Perkin Elmer) on a 10 g/mL fibronectin coating (F1141, Sigma). Media was changed 4 hours after seeding and every two days until transfection. Seeding and media changes were performed by a Zephyr Liquid Handler from Perkin Elmer.

Cells were transfected with the mirVana™ micro-RNA Mimic Library (Pre-defined Human v 21, 4464074, ThermoFisher) using Lipofectamine RNA iMax (13778-150, ThermoFisher) in OptiMEM medium (11058-21, Gibco). This library is composed of 2565 mimic human microRNAs. We used 25 nM as the final concentration of miRNAs. Twenty-four hours after transfection, medium was completely changed and 72 hours after transfection, bright field imaging of the cells was performed with an automated high content screening system (Cell voyager CV8000, Yokogawa) with bright field at 37° C. and 5% CO2. A 10-second movie was recorded for each well at a framerate of 37 images per second using brightfield light microscopy with a binning of 2, generating 500×500 pixels images.

To analyze raw image sequences of hiPSC-CM contraction/relaxation, we implemented an optical vector flow script (27). The magnitude of each vector was then computed and integrated over the entire image, providing a total contraction amplitude per frame. The final signal was then created using the 357 amplitude values sequentially.

Plates' optical vector flow signals were analyzed with a custom-made Matlab script (MatWorks). In this script, several readouts are extracted: the amplitude of contraction, the beating frequency, maximum and mean velocities, the contraction-time integral (Area under the curve), the time to contract from 10 to 90% of amplitude, the time to relax from 90% to 10% and the peak duration.

We analyzed plates results with the open access software HCS analyzer (28). The assay was performed in three independent replicates. Hits were selected according to their Z score on the three replicates: hit were validated when the Z score was above 2 in at least 2 replicates and the mean Z score of the three replicates above 2. Z score was calculated with the following equation:

$$Z = \frac{x - μ}{σ}$$

Where μ is the mean of the mean relaxation velocity on the plate, x the mean relaxation velocity of the miRNA, and σ the standard deviation of the plate.

hiPSC-CM Based Engineered Cardiac Tissues
hiPSC Culture and Differentiation

For 3D culture, we used the SKiPSC-31.3 hiPSC cell line, derived from human dermal fibroblasts from a healthy 45-year-old volunteer as previously published (29). The hiPSCs cells were seeded on Matrigel and cultured in mTeSR1 medium (Stemcell Technologies). When hiPSCs reached a confluency of 70%-80%, cells were passaged in clumps by scraping with a pipette tip. A medium change was performed every 24 hours. Cultures were maintained at 37° C. in a humidified incubator with 5% CO2. The hiPSC line used in this study was assessed for pluripotency and routinely tested for mycoplasma.

Once confluent, the hiPSC cells were differentiated into cardiomyocytes using a small molecule-modulated differentiation and glucose starvation (30). Briefly, mTeSR1 medium (Stemcell Technologies) was changed by RPMI supplemented with B27 without insulin (ThermoFisher Scientific) and 6 μM CHIR-99021 (Abcam), and maintained in a 37° C. and 5% CO2 incubator for 48 h. The medium was changed to RPMI-B27 without insulin for 24 hours, and then to RPMI-B27 without insulin supplemented with 5 μM IWR-1 (Sigma) for 48 hours. On day 5, the medium was changed back to RPMI-B27 without insulin for 48 hours. From day 7 onwards, cells were placed in RPMI-B27 with insulin and media change every two days. At day 11 the medium was changed to low glucose medium for 3 days. Cardiomyocytes were then replated in RPMI-B27 with insulin. At day 15, medium is changed for a second glucose deprivation for three more days. Starting from day 18, medium is changed every two days with RPMI-B27 with insulin.

To assess the differentiation efficiency, at day 21, cells were strained with an APC anti-cardiac troponin T (TNNT2) antibody (130-106-689, Miltenyi Biotech; 1:100) or APC isotype control (130-104-615, Miltenyi Biotech; 1:100) and analyzed by flow cytometry.

### Human Fibroblasts Culture

We use a commercially available human fibroblast cell line from Lonza (CCC2511, lot 4888388). Fibroblasts were cultured in T75 flasks and maintained in DMEM supplemented with 10% FBS and 1% penicillin-streptomycin. Cells with low passage number (<7) were used.

hiPSC-CM Based Engineered Cardiac Tissue

To build Engineered Cardiac Tissue (ECT) we prepared a mix per tissue of 1.2 million hiPSC-CMs, 0.3 million fibroblasts, 2 mg/mL collagen I (354249, Corning) and 0.9 mg/mL Matrigel (356231, Corning) in a HEPES/MEM media. On day 22 of differentiation, cardiomyocytes were dissociated with enzymatic digestion (130-110-204, Miltenyi) and fibroblasts with TrypLE Express Enzyme (12605028, ThermoFisher).

The cell-matrix mix (100 μL/mold) was seeded in a flexible PDMS mold and placed at 37° C. and 5% CO2 (25, 47, 48). After two hours, ECTs were fed with DMEM supplemented with 10% FBS, 1% Penicillin-streptomycin, and a calcium concentration of 2.3 mM. Medium was changed every two days. After 13 days of culture, contractile forces were measured just before transfection. Forward transfection was performed using 25 nM of microRNA (miRNA negative control or hsa-miR-548v) in OptiMEM using Lipofectine RNAimax. Medium was changed 24 hours after transfection and contractile forces were measured 72 hours after transfection.

Contractile force measurements were captured with a high-speed CCD camera (PL-D672MU, Pixelink) while custom LabVIEW software developed by K. Costa's lab (31) tracked the centroid movement of the tips of the flexible posts. Force was converted from the deflection of the PDMS posts by an elastic beam-bending equation (31). A custom MATLAB script, similar to the one developed for the HCS campaign, was used to extract several readouts, including the developed force and mean relaxation velocity.

### Quantitative PCR

RNA and microRNAs were extracted from ECTs using QIAzol lysis Reagent and purified with the miRNeasy mini kit (217004, Qiagen), as per the manufacturer's instructions. Then, 10 ng of extracted RNA and microRNAs was subjected to reverse transcription using the miRCURY LNA RT Kit (339306, Qiagen) as per the manufacturer's instructions. The resulting cDNA was subjected to qPCR using SYBR Select Master Mix (4472908, Applied Biosystems) on Quant Studio 3 Real-Time PCR system (Thermo Fisher) as per the following condition: 95° C. for 2 min, 40 cycles of 95° C. for 10 s and 56° C. for 1 min, followed by 95° C. for 10 s and 60° C. for 1 min. The relative expression of hsa-miR-548v was calculated using the comparative cycle threshold (Ct) method. The ΔCt was calculated by subtracting RNU1A1 Ct from hsa-miR-548v Ct whereas ΔΔCt was obtained by subtracting the mean ΔCt of ECT transfected with miR negative control from ΔCt of the sample.

hiPS-CM Single Cell Distensibility Measurements
hiPS-CM Micropatterning

To promote rod-shaped cardiomyocytes, we used micropatterning technology (4DCell, Montreuil, France). 35 days after differentiation, cardiomyocytes were seeded in micropatterned coverslips with a rectangular shape (custom-made, size: 120 μm×30 μm). The micropatterned substrate allows cells to adhere only on micrometer-sized defined region. Cells were cultured for 5 days on micropatterned slides before forward transfection.

### Distensibility Measurements

72 hours after transfection, cells were stretched in order to evaluate their distensibility properties, using the Myostretcher system from Ionoptix. This system is composed of two micromanipulators connected to an optical force transductor (OptiForce, Ionoptix) and a piezoelectric length controller (motor). Cells were stretched to different lengths in a staircase protocol.

### Cell Attachment Procedure

Micropatterned cells were enzymatically dissociated with type II collagenase (50 U/mL) for 20 minutes at 37° C. The cell was glued on the Myostretcher's tips using a biological adhesive material (Myotak, Ionoptix) at its two distal edges. To fully detach the cell from the slide, we used slight lateral movements as previously described (26) and lift the cell to start staircase protocol.

### Transcriptome-Wide Analysis

To perform RNA sequencing we used iCell cardiomyocytes² from FCDI. After 6 days of culture, we performed forward transfection of miRNA and extract RNA 3 days after transfection.

### Quantification of Gene Expression

STAR was used to obtain the number of reads associated to each gene in the Gencode v 31 annotation (restricted to protein-coding genes, antisense and lincRNAs). Raw counts for each sample were imported into R statistical software. Extracted count matrix was normalized for library size and coding length of genes to compute FPKM expression levels.

### Unsupervised Analysis

The Bioconductor edgeR package was used to import raw counts into R statistical software, and compute normalized log 2 CPM (counts per millions of mapped reads) using the TMM (weighted trimmed mean of M-values) as normalization procedure. The normalized expression matrix from the 1000 most variant genes (based on standard deviation) was used to classify the samples according to their gene expression patterns using principal component analysis (PCA), hierarchical clustering and consensus clustering. PCA was performed by FactoMineR::PCA function with “ncp=10, scale.unit=FALSE” parameters. Hierarchical clustering was performed by stats::hclust function (with euclidean distance and ward.D method). Consensus clustering was performed by ConsensusClusterPlus::ConsensusClusterPlus function to examine the stability of the clusters. We established consensus partitions of the data set in K clusters (for K=2, 3, . . . , 8), on the basis of 1,000 resampling iterations (80% of genes, 80% of sample) of hierarchical clustering, with euclidean distance and ward.D method. Then, the cumulative distribution functions (CDFs) of the consensus matrices were used to determine the optimal number of clusters (K=3 for instance), considering both the shape of the functions and the area under the CDF curves. tSNE analysis was performed with the Bioconductor Rtsne package applied to the PCA object (theta=0.0, perplexity=, max_iter=1000).

### Differential Expression Analysis

The Bioconductor edgeR package was used to import raw counts into R statistical software. Differential expression analysis was performed using the Bioconductor limma package and the voom transformation. To improve the statistical power of the analysis, only genes expressed in at least one sample (FPKM>=1) were considered. A qval threshold of <=0.05 and a minimum fold change of 2 were used to define differentially expressed genes. The enrichment for gene sets and canonical pathways (KEGG and GO terms) on significantly down-expressed genes was analyzed using the metascape web-tool (32).

### Calcium Transient Analysis

After imaging cells with the HCS system, cells were loaded with Fluo4 Direct Calcium Assay kit (F10471, ThermoFisher, 0.5×final concentration). Then, cells were incubated 30 minutes at 37° C. followed by 30 minutes at room temperature. Calcium imaging was performed using the functional drug screen system (FDSS) from Hamamatsu for 2 minutes. Signals analysis was then performed using WaveAnalysis Software from Hamamatsu.

### Immunostaining and Imaging

After 7 days of culture, hiPSC-CMs were sequentially fixed with 4% paraformaldehyde (PFA) (1573590, Electron Microscopy Sciences) for 10 min and then permeabilized and blocked with 0.5% Triton X-100 (T-8787, Sigma), 2% bovine serum albumin (BSA) (001-000-162, Jackson ImmunoResearch) in PBS (blocking solution) for 1 hour. Subsequently, primary antibody incubation was performed overnight at 4° C. in 1:10 diluted blocking solution: Cardiac-TroponinT (ab45932, Abcam; 1:500), Alpha-Actinin (A7811, Sigma Aldrich; 1:1000), Alpha-Tubulin (ab7291, Abcam; 1:200). After washing, cells were incubated with secondary antibodies goat anti-mouse, anti-rabbit or anti-rat immunoglobulin G conjugated to Alexa Fluor 488 (A10680, ThermoFisher; 1:500) (712-545-153, Jackson ImmunoResearch; 1:500) or 546 (A11010, ThermoFisher; 1:500)/DAPI (ThermoFisher), and mounted with Dako Faramount Aqueous Mounting (S3025, Agilent). Fluorescent images were captured on a Leica SPE confocal microscope at 63× objectives as appropriate. Image processing and analysis were performed using ImageJ.

### Results

Screening for miRNAs Regulating CM Relaxation Velocities

We performed a high-content, microscopy-based, high-throughput screening in human induced pluripotent stem cells derived cardiomyocytes (hiPSC-CM) using a library of 2565 human miRNA mimics (miRbase sequence database version 21) (data not shown). The miRNA mimics were transfected to the cultures of hiPSC-CM (forward transfection) which presented as beating monolayers in 384-well plates. Three days later, we recorded high-speed movies of iPSC-CM beating monolayers in each well using an automated high-content screening microscope. The image sequences were then analyzed by optical vector flow analysis with a high-performance computer (HPC) in order to model the hiPSC-CM contractile movements and measure the relaxation and contraction velocities (data not shown). The screening was performed in triplicate. In addition to the miRNA negative control, a random sequence that has been tested in human cell lines and proven not to produce identifiable effects, we integrated 3 miRNAs mimics in each plate as quality control. We also tested different miRNAs concentration to assess the impact of transfection on our readouts (data not shown).

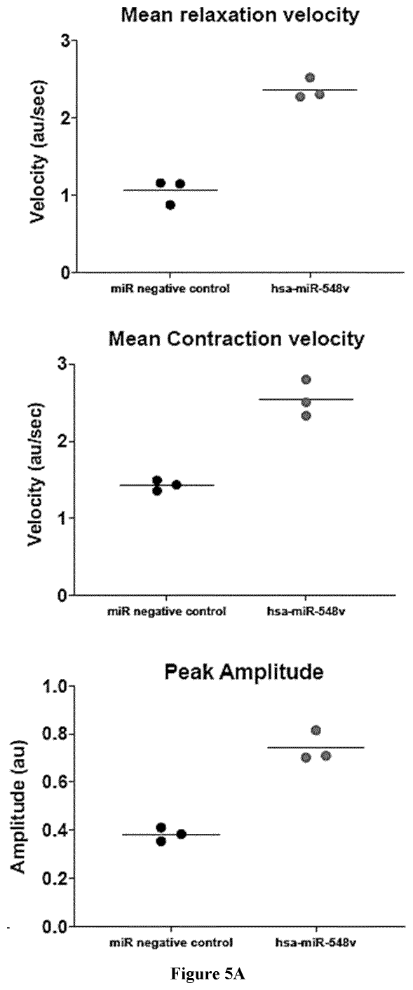

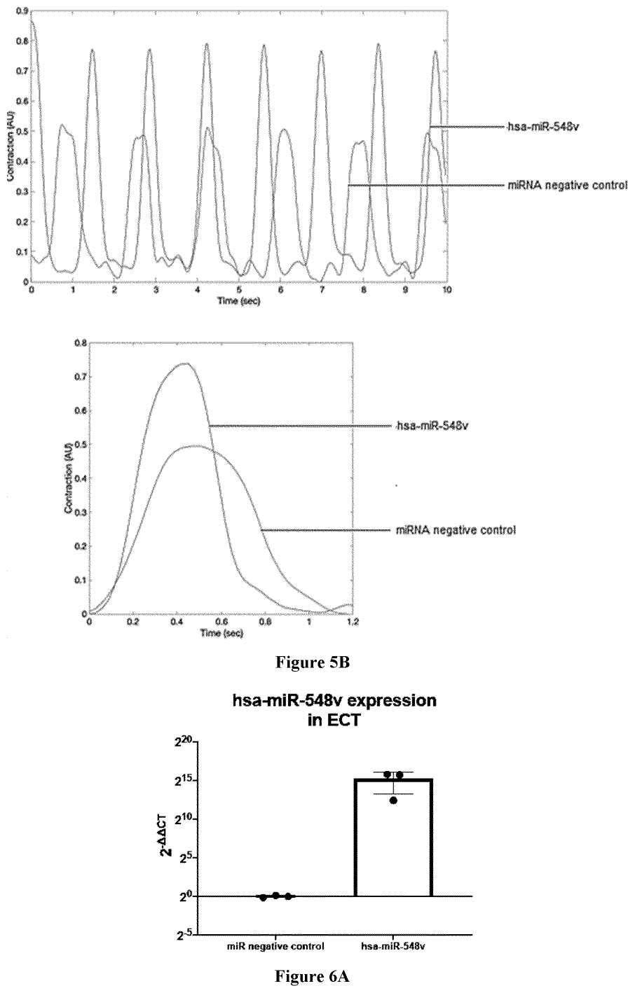

As compared to hiPSC-CMs treated with control miRNA, 144 miRNAs accelerated the mean relaxation velocities in at least one of the three independent screen replicates (Z score>2, p-value<0.05) (data not shown), but 10 miRNAs increased significantly the relaxation velocity in at least 2 independent replicates (data not shown). The maximal and most reproducible changes in relaxation phase were observed with hsa-mir-548v, which significantly increased the relaxation velocities in the three independent screen replicates (data not shown). Similar results were obtained when considering the maximal relaxation velocities. In addition to its impact on relaxation, hsa-miR-548v also increased contraction velocities, beating amplitude and rate (FIG. 5A), suggesting a global improvement in cardiomyocytes' mechanics (FIG. 5B).

hsa-miR-548v is part of the large primate-specific miR-548 family and is located on chromosome 8. The miR-548 superfamily is the largest miRNA family in the human genome with 74 miRNAs members. A down regulation of at least 10 miRNA-548 family members was identified by genome-wide analysis on peripheral blood mononuclear cells (PBMCs) from patient with heart failure with reduced ejection fraction (21). However, little is known about the implication of hsa-miRNA-548v in cardiovascular disorders. We used Tissue atlas2 (22), a small noncoding RNA expression tissue atlas determined from humans, to explore hsa-miR-548v expression in human organs and found very low levels of expression in any of the 21 organs explored (data not shown). Moreover, Fantom5 (23), shows an enrichment of hsa-miR-548v in endothelial cells (data not shown). These data suggest that there is no or limited basal expression of hsa-miR-548v in cardiomyocytes or fibroblasts.

The results of this HC-screening thus indicated hsa-miR-548v transfer as having interesting lusitropic effects which prompted us to further investigate its impact in cardiomyocytes.

### Hsa-Mir-548v Improves Cardiac Relaxation at the Tissue Level

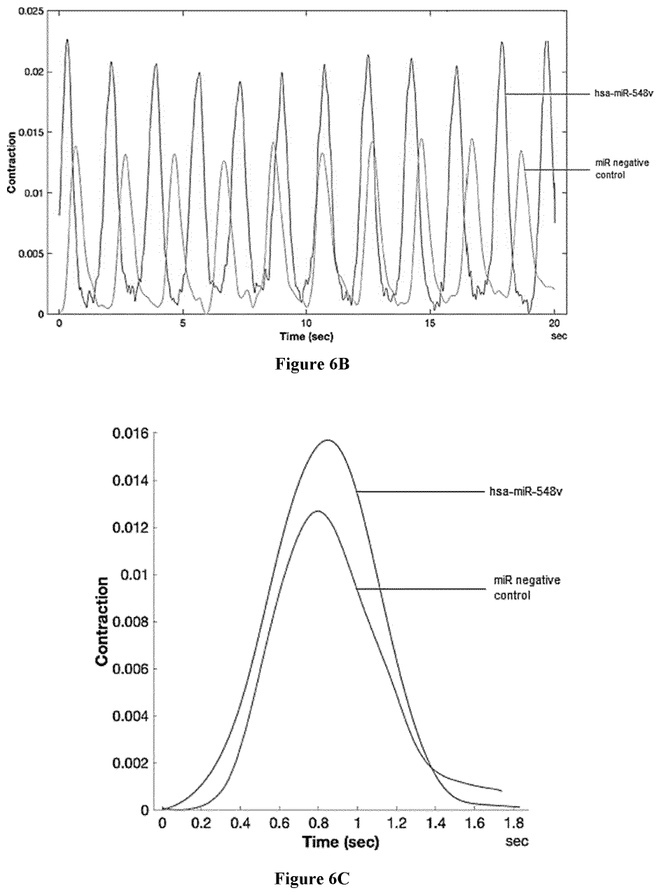

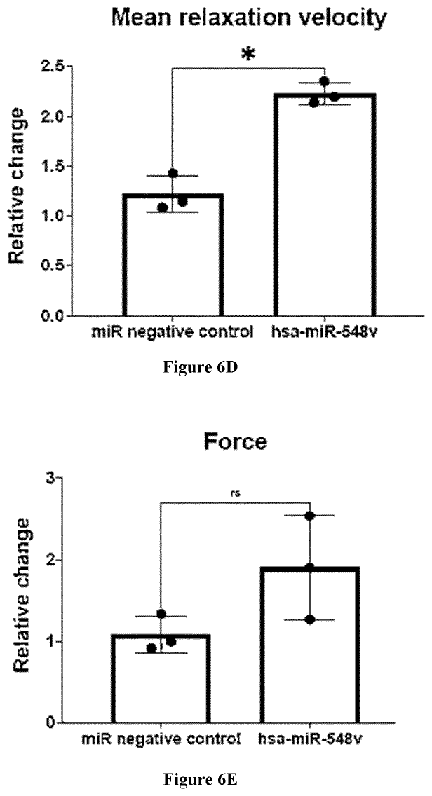

The function of cardiomyocytes depends on several parameters in their 3D environment, including the extracellular matrix and the multicellular interactions. Furthermore, hiPSC-CM display a more mature phenotype in 3D organoids as compared to 2D-monolayer culture (24). To further characterize the effects of hsa-miR-548v on cardiac function, we tested its impact on hiPSC-CM engineered cardiac tissues (hECT). We used a previously reported 3D platform (25) composed by a 4:1 ratio of hiPS-CM:fibroblasts, embedded in a collagen and Matrigel matrix, and that form a structure similar to a trabecular cardiac muscle (data not shown). We also tested a different iPSC line than used in the HCS (data not shown) with a differentiation protocol that led to the production of more than 90% TNNT2 positive cells (data not shown). After 13 days of culture and just before miRNA transfection, hECT were recorded with a high-speed camera to capture their motion and assess their contractility (data not shown). In line with predicted expression from public databases, we found no basal expression of hsa-miR-548v expression in control hECT. Three days after transfection, we found hsa-mir-548v expression in hECT transfected with hsa-miR-548v and none in hECT transfected with miR negative control (FIG. 6A). hECT were recorded again (data not shown) and the post-transfection parameters were used to evaluate the relative changes on relaxation velocity and force under paced conditions (data not shown). FIGS. 6B and 6C show representative signals of hECT 3 days after transfection. Concordant with the HCS results, relaxation velocities of hECT transfected with hsa-miR-548v were more than doubled after transfection as compared to hECT transfected with miR negative control (FIGS. 6C and 6D). There was a non-significant trend for a higher developed force in hECT transfected with hsa-miR-548v (FIG. 6E). These data suggest that hsa-miR-548v transfer improves cardiac lusitropy in a multi-cellular environment formed at the tissue level, and reproduces its benefit on relaxation in different iPS-CM cell lines.

Hsa-Mir-548v does not Change Calcium Transients

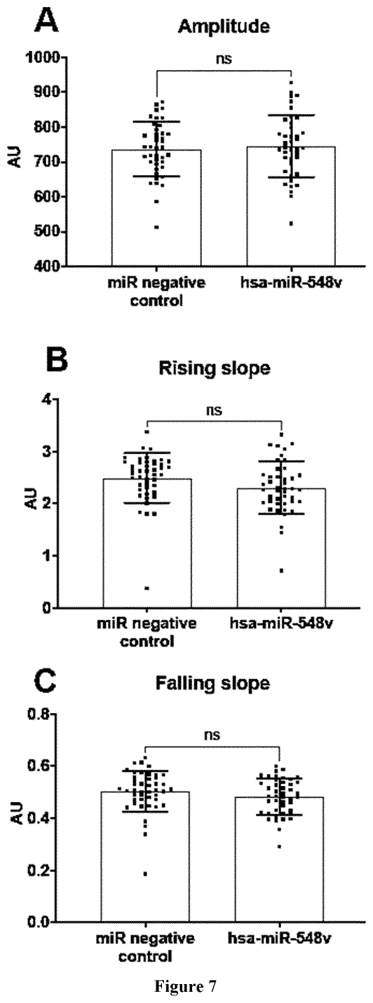

As calcium is an important contributor to relaxation rate and force development in cardiomyocytes, we next determined whether hsa-miR-548v is affecting calcium handling in hiPSC-CMs. We recorded the basal intracellular Ca2+([Ca2+]i) transients of iPSC-CMs WT-CMs treated with hsa-miR-548v or miR negative control and loaded three days later with fluo-4, a Ca2+ indicator (data not shown). We found no significant changes in calcium transients' amplitudes (FIG. 7A, data not shown), nor in release and reuptake kinetics parameters (FIGS. 7B and 7C), whereas relaxation velocities were significantly improved in hsa-miR-548v treated hiPSC-CMs concordant with our previous results (data not shown). Overall, these results indicate that hsa-miR-548v significantly changed cardiomyocytes' lusitropy without involving changes in Ca2+ handling properties of hiPSC-CMS.

Hsa-miR-548v Changes the Expression of Intracellular Components Associated with Mechanotransduction

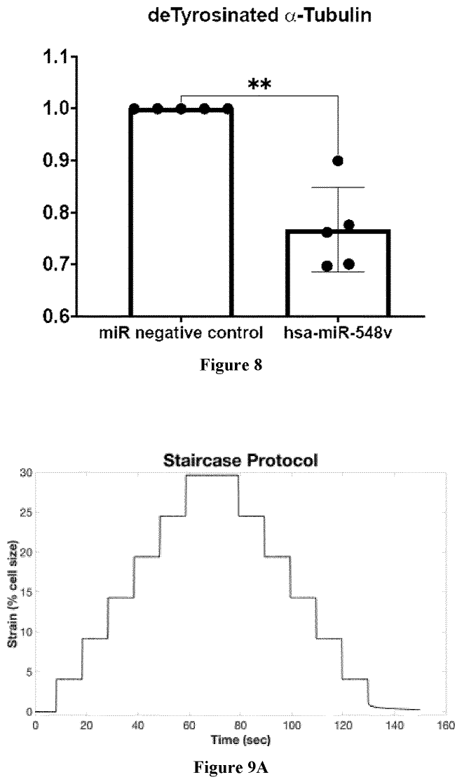

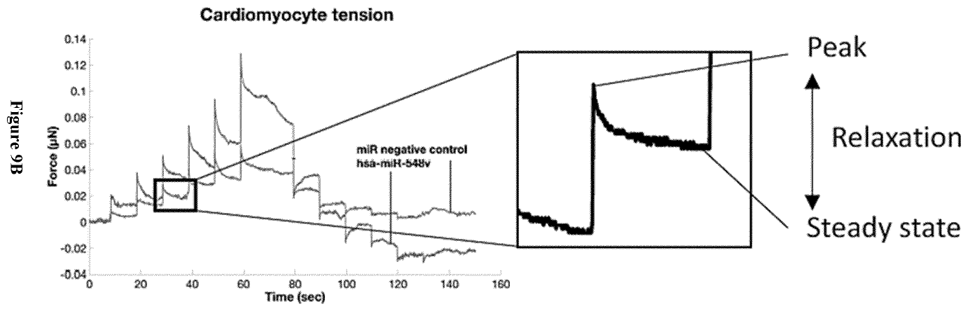

These results prompted us to investigate other processes that can regulate cardiac relaxation. We thus assessed global transcriptome changes by deep-sequencing hiPSC-CMs RNA after transfection with hsa-miR-548v vs. miR negative control (data not shown). The clustering analysis revealed distinct profiles between groups (data not shown) with 645 down-regulated transcripts (FPKM>1 and >2.0-fold down-regulation) and 365 up-regulated transcripts (FPKM>1 and >2.0-fold up-regulation)(data not shown). We first observed that NPPB (encoding for the natriuretic peptide B, a well-known hormone secreted by cardiac ventricular myocytes in response to myocardial stretch) is the most down-regulated gene in response to hsa-miR-548v (log 2 fold change −4.02, q-value 4.8×10-17, data not shown). We further analyzed the datasets of down-regulated transcript to assess sets of genes encoding for cardiomyocytes' intra-cellular components that typically contribute to myocardial elasticity (i.e., calcium handling, microtubule network, filaments and cytoskeletal proteins)(data not shown). We found a significant down-expression of DESMIN (log 2 fold change −1.57, q-value 3.2×10-12)(data not shown), the predominant intermediate filament (data not shown), as well as two important mechano-sensors, cardiac ankyrin repeat proteins ANKRD1/CARP1 (log 2 fold change −2.76, q-value 1.6×10-16) and ANKRD2/CARP2 (log 2 fold change −3.06, q-value 4.6×10-8) (data not shown). CARPs are highly expressed in cardiomyocytes where they interact with the intermediate filament (desmin) proteins, acting as an important regulator of the stretch-sensing machinery. Other components showed lower differences between groups (data not shown). Major tubulin isotypes expressions tend to increase. As detyrosinated microtubules increase myocyte contractile duration (13), we evaluated the level of detyrosinated α-tubulin in hiPSC-CM transfected with hsa-miR-548v compared to the ones transfected with the miR negative mimic and found a significant reduction of this microtubules post-translational modification in hsa-miR-548v transfected cells (FIG. 8). This is in line with previous studies showing that suppression of detyrosinated microtubules improves relaxation time in myocytes (13, 26).

Metascape and gene set enrichment analysis (GSEA) revealed that the 645 most downregulated genes were enriched in cardiovascular networks as “heart development” or “circulatory system process”, in line with a previous in-silico study indicating a significant association between members of miRNA-548 family and cardiovascular system development (21). In addition, further analyses for enriched canonical pathways revealed a down-regulation of multiple members and regulators of the Mitogen-Activated Proteins Kinases (MAPK) signaling cascade (data not shown) which typically serve as specialized transducer of stress response. Overall, these results indicate that hsa-miR-548v dysregulated multiple targets, including structural components implicated in the transmission of mechanical forces and the resistance to cyclic deformation.

Hsa-miR-548v Impacts the Internal Distensibility Properties of Human iPSC-Derived Cardiomyocytes at the Single-Cell Level

We next studied the mechanical properties of single-cell human hiPSC-derived cardiomyocytes transfected with hsa-miR-548v or miR negative control (data not shown). The measure of mechanical properties requires typical rod-shape morphology as observed in isolated cardiomyocytes from rodent and human adult hearts, whereas isolated hiPSC-CM appears to have a rounded morphology. We thus developed a protocol to generate hiPSC-CM on specifically-designed micropatterned slides with a rod-shape morphology (data not shown). Briefly, the slides are stamped with Matrigel-coated rectangular islands (120 μm×30 μm) surrounded with an anti-adhesive agent (PMOXA). After seeding, hiPSC-CM grow in the pre-designed areas and reach a rod-shaped morphology (data not shown). This method also imposes a constant size of cardiomyocytes, and background stiffness limiting variability (data not shown).

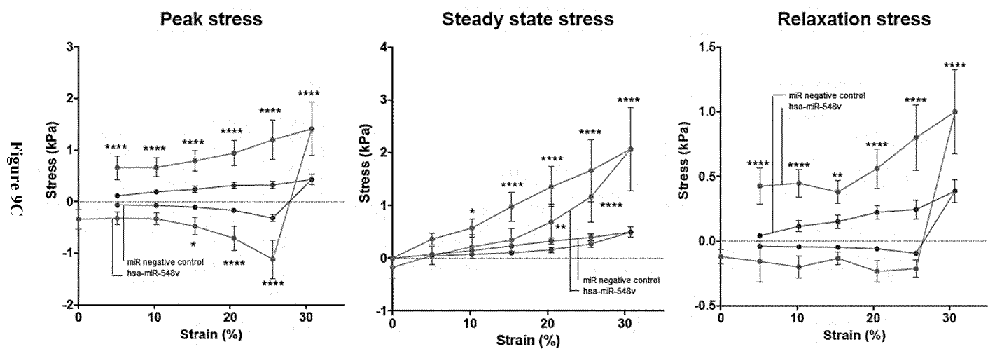

We use a similar protocol as Ballan and colleagues (26) to attach the cells, and were able to successfully stretch the cells up to 40% of their initial length (data not shown). To assess length-tension relationship, we used a stair case protocol, in which a piezo-electric motor is moving gradually of 6 m with a 62.5 μm/sec stretch velocity, every 10 seconds (FIG. 9A). Force measurements were held without any contraction inhibitors. Both cardiomyocytes transfected with miR negative control or hsa-mir-548v showed a positive length-tension relationship as the forces increased with cell stretching (Frank-Starling law)(FIG. 9B). Viscous tension (Relaxation stress) increased non linearly with stretch increasing. We observed a significant increase of effective (peak stress) and resting (steady state stress) tension of cardiomyocytes transfected by hsa-miR-548v compared to the ones transfected by miR negative control (FIG. 9C) and these difference between groups was strikingly growing as the stretch was increasing. Viscoelastic tension was increased during stretch of hsa-miR-548v transfected cardiomyocytes compared with miR negative control transfected cardiomyocytes. hsa-miR-548v increases both elastic tension (P<0.001) and a viscous tension (P=0.0004). This data shows that hsa-miR-548v impacts viscoelastic response to stretch by increasing both its elastic and viscous tension.

### REFERENCES

Throughout this application, various references describe the state of the art to which this invention pertains. The disclosures of these references are hereby incorporated by reference into the present disclosure.
1. Seko Y, Kato T, Shiba M, Morita Y, Yamaji Y, Haruna Y, et al. Association of the low e′ and high E/e′ with long-term outcomes in patients with normal ejection fraction: a hospital population-based observational cohort study. BMJ Open. 2019; 9(11):e032663.
2. Borlaug B A, Jaber W A, Ommen S R, Lam C S, Redfield M M, and Nishimura R A. Diastolic relaxation and compliance reserve during dynamic exercise in heart failure with preserved ejection fraction. Heart. 2011; 97(12):964-9.
3. Poliner L R, Dehmer G J, Lewis S E, Parkey R W, Blomqvist C G, and Willerson J T. Left ventricular performance in normal subjects: a comparison of the responses to exercise in the upright and supine positions. Circulation. 1980; 62(3):528-34.
4. Cheng C P, Igarashi Y, and Little W C. Mechanism of augmented rate of left ventricular filling during exercise. Circ Res. 1992; 70(1):9-19.
5. Phan T T, Abozguia K, Nallur Shivu G, Mahadevan G, Ahmed I, Williams L, et al. Heart failure with preserved ejection fraction is characterized by dynamic impairment of active relaxation and contraction of the left ventricle on exercise and associated with myocardial energy deficiency. J Am Coll Cardiol. 2009; 54(5):402-9.
6. Haykowsky M J, Brubaker P H, John J M, Stewart K P, Morgan™, and Kitzman D W. Determinants of exercise intolerance in elderly heart failure patients with preserved ejection fraction. J Am Coll Cardiol. 2011; 58(3):265-74.
7. Caporizzo M A, and Prosser B L. Need for Speed: The Importance of Physiological Strain Rates in Determining Myocardial Stiffness. Front Physiol. 2021; 12:696694.
8. Caporizzo M A, Chen C Y, Bedi K, Margulies K B, and Prosser B L. Microtubules Increase Diastolic Stiffness in Failing Human Cardiomyocytes and Myocardium. Circulation. 2020; 141(11):902-15.
9. Granzier H L, and Irving T C. Passive tension in cardiac muscle: contribution of collagen, titin, microtubules, and intermediate filaments. Biophysical Journal. 1995; 68(3):1027-44.
10. Linke W A. Titin Gene and Protein Functions in Passive and Active Muscle. Annu Rev Physiol. 2018; 80:389-411.
11. Chung C S, Methawasin M, Nelson O L, Radke M H, Hidalgo C G, Gotthardt M, et al. Titin based viscosity in ventricular physiology: an integrative investigation of PEVK-actin interactions. J Mol Cell Cardiol. 2011; 51(3):428-34.
12. Herwig M, Kolijn D, Lodi M, Holper S, Kovacs A, Papp Z, et al. Modulation of Titin-Based Stiffness in Hypertrophic Cardiomyopathy via Protein Kinase D. Front Physiol. 2020; 11:240.
13. Chen C Y, Caporizzo M A, Bedi K, Vite A, Bogush A I, Robison P, et al. Suppression of detyrosinated microtubules improves cardiomyocyte function in human heart failure. Nat Med. 2018; 24(8):1225-33.
14. Robison P, Caporizzo M A, Ahmadzadeh H, Bogush A I, Chen C Y, Margulies K B, et al. Detyrosinated microtubules buckle and bear load in contracting cardiomyocytes. Science. 2016; 352(6284):aaf0659.
15. Rainer P P, Dong P, Sorge M, Fert-Bober J, Holewinski R J, Wang Y, et al. Desmin Phosphorylation Triggers Preamyloid Oligomers Formation and Myocyte Dysfunction in Acquired Heart Failure. Circ Res. 2018; 122(10):e75-e83.
16. Agnetti G, Herrmann H, and Cohen S. New roles for desmin in the maintenance of muscle homeostasis. FEBS J. 2021.
17. Brodehl A, Hain C, Flottmann F, Ratnavadivel S, Gaertner A, Klauke B, et al. The Desmin Mutation DES-c.735G>C Causes Severe Restrictive Cardiomyopathy by Inducing In-Frame Skipping of Exon-3. Biomedicines. 2021; 9(10).
18. Brodehl A, Pour Hakimi S A, Stanasiuk C, Ratnavadivel S, Hendig D, Gaertner A, et al. Restrictive Cardiomyopathy is Caused by a Novel Homozygous Desmin (DES) Mutation p. Y122H Leading to a Severe Filament Assembly Defect. Genes (Basel). 2019; 10(11).
19. Friedman R C, Farh K K, Burge C B, and Bartel D P. Most mammalian mRNAs are conserved targets of microRNAs. Genome Res. 2009; 19(1):92-105.
20. Lemons D, Maurya M R, Subramaniam S, and Mercola M. Developing microRNA screening as a functional genomics tool for disease research. Front Physiol. 2013; 4:223.
21. Gupta M K, Halley C, Duan Z H, Lappe J, Viterna J, Jana S, et al. miRNA-548c: a specific signature in circulating PBMCs from dilated cardiomyopathy patients. J Mol Cell Cardiol. 2013; 62:131-41.
22. Keller A, Groger L, Tschernig T, Solomon J, Laham O, Schaum N, et al. miRNATissueAtlas2: an update to the human miRNA tissue atlas. Nucleic Acids Res. 2022; 50(D1):D211-D21.
23. de Rie D, Abugessaisa I, Alam T, Amer E, Amer P, Ashoor H, et al. An integrated expression atlas of miRNAs and their promoters in human and mouse. Nat Biotechnol. 2017; 35(9):872-8.
24. Seguret M, Vermersch E, Jouve C, and Hulot J S. Cardiac Organoids to Model and Heal Heart Failure and Cardiomyopathies. Biomedicines. 2021; 9(5).
25. Turnbull I C, Karakikes I, Serrao G W, Backeris P, Lee J J, Xie C, et al. Advancing functional engineered cardiac tissues toward a preclinical model of human myocardium. FASEB J. 2014; 28(2):644-54.
26. Ballan N, Shaheen N, Keller G M, and Gepstein L. Single-Cell Mechanical Analysis of Human Pluripotent Stem Cell-Derived Cardiomyocytes for Drug Testing and Pathophysiological Studies. Stem Cell Reports. 2020; 15(3):587-96.
27. Brox T, Bruhn A, Papenberg N, and Weickert J. High Accuracy Optical Flow Estimation Based on a Theory for Warpingâ<†. Computer Vision. 2004; ECCV 2004 Volume 3024:25â€″36.
28. Ogier A, and Dorval T. HCS-Analyzer: open source software for high-content screening data correction and analysis. Bioinformatics. 2012; 28(14):1945-6.
29. Karakikes I, Senyei G D, Hansen J, Kong C W, Azeloglu E U, Stillitano F, et al. Small molecule-mediated directed differentiation of human embryonic stem cells toward ventricular cardiomyocytes. Stem Cells Transl Med. 2014; 3(1):18-31.
30. Sharma A, Li G, Rajarajan K, Hamaguchi R, Burridge P W, and Wu S M. Derivation of highly purified cardiomyocytes from human induced pluripotent stem cells using small molecule-modulated differentiation and subsequent glucose starvation. J Vis Exp. 2015(97).
31. Serrao G W, Turnbull I C, Ancukiewicz D, Kim D E, Kao E, Cashman T J, et al. Myocyte-depleted engineered cardiac tissues support therapeutic potential of mesenchymal stem cells. Tissue Eng Part A. 2012; 18(13-14):1322-33.
32. Zhou Y, Zhou B, Pache L, Chang M, Khodabakhshi A H, Tanaseichuk O, et al. Metascape provides a biologist-oriented resource for the analysis of systems-level datasets. Nat Commun. 2019; 10(1):1523.

1. A method for improving striated muscle cell relaxation in a subject in need thereof comprising administering to the subject a therapeutically effective amount of at least one miRNA selected from the group consisting of miR-548u and miR-548v.

2. The method according to claim 1 wherein a nucleotide sequence of miR-548u and miR-548v is at least about 80%, 85%, 90%, 95%, 98%, 99% or more identical to a nucleotide sequence of miR-548u or miR-548v.

3. The method of claim 1 wherein the at least one miRNA is miR-548u and comprises the stem loop sequence as set forth in SEQ ID NO:3.

4. The method of claim 1 wherein the at least one miRNA is miR-548v and comprises the stem loop sequence as set forth in SEQ ID NO:4.

5. The method according to claim 1 wherein the subject suffers from striated muscle stiffness.

6. The method according to claim 5 wherein the at least one miRNA is administered in combination with a muscle relaxant.

7. The method according to claim 6, wherein the muscle relaxant is atracurium besilate, baclofene, carisoprodol, cisatracurium besilate, dantrolene, mivacurium chlorure, methocarbamol, pancuronium bromure, rocuronium bromure, suxamethonium, thiocolchicoside, tizanidine, tetrazepam or vecuronium bromure.

8. The method according to claim 5, wherein the striated muscle stiffness is induced by Parkinson's disease.

9. The method according to claim 8, wherein the at least one miRNA is administrated in combination with a dopamine precursors, a dopamine agonists or an inhibitor of dopamine precursor degradation.

10. The method according to claim 1 wherein the striated muscle cell is a cardiomyocyte.

11. The method according to claim 10 wherein the subject suffers from heart failure with preserved ejection fraction.

12. The method according to claim 11, wherein the at least one miRNA is administrated in combination with Angiotensin Converting Enzyme Inhibitors (ACEIs), angiotensin, Aldosterone Receptor Antagonists (ARDs) or β-blockers.

13. A therapeutic composition comprising at least one miRNA selected from the group consisting of miR-548u or miR-548v, wherein the therapeutic composition is in a form that is suitable for administration to a subject in need of improved striated muscle relaxation.
